(19) United States
(12) Patent Application Publication
(10) Pub. No.: US 2026/0115459 A1
(43) Pub. Date: Apr. 30, 2026

(54) IMPLANT USABLE FOR PACING AND INCLUDING HYDROGEL CAPABLE OF CURING IN SITU, A SYSTEM AND A METHOD FOR DELIVERING SUCH IMPLANT TO A PATIENT

(71) Applicant: Texas Heart Institute, Houston, TX (US)

(72) Inventors: Mathews Medayil JOHN, Katy, TX (US); Allison D. POST, Houston, TX (US); Lukas Michael JAWORSKI, Pearland, TX (US); Drew Hunter Bernard, Houston, TX (US); Mehdi Razavi, Houston, TX (US)

(21) Appl. No.: 19/293,677

(22) Filed: Aug. 7, 2025

Related U.S. Application Data
(60) Provisional application No. 63/704,377, filed on Oct. 7, 2024.

(51) Int. Cl. A61N 1/05; A61N 1/362; A61N 1/372

(52) U.S. Cl. CPC A61N 1/057; A61N 1/362; A61N 1/372; A61N 2001/0578

(57) ABSTRACT

An implantable an implantable shaft system, having a proximal connector, conductive wires, and lumens capable of injecting precursor solutions into a patient. The system also includes a distal mixing head capable of mixing the precursor solutions in the patient to form a hydrogel in situ that is in contact with an electrically conductive distal electrode. The system has pinching and/or detachment mechanisms to close and seal the lumens to allow retrieval a portion of the system from the patient, leaving only the implant in the patient that can be connected to a pacemaker or other device. One or more occluder assemblies may be used to anchor the shaft system at a target location and control the placement of the hydrogel. The system may also use a detachable container for storing precursor solutions, a catheter hub, and/or a catheter hub for connecting the implant to the detachable container.

### RELATED APPLICATION

This application claims the benefit under 35 U.S.C. § 119(e) of U.S. provisional application 63/704,377, filed Oct. 7, 2024, which is incorporated by reference herein in its entirety.

### GOVERNMENT FUNDING

This invention was made with government support under R01HL162741 awarded by the National Institutes of Health. The government has certain rights in the invention.

### FIELD OF THE INVENTION

The application relates generally to an implant usable for providing an electrical stimulus (i.e., for pacing), wherein the implant includes hydrogel that is capable of curing in situ (i.e., in the body of the patient), a system for delivering such an implant to a patient, and a method of delivery of such an implant. The application relates more particularly to an implant that is usable for heart pacing, a system for delivering such an implant in or near the heart of a patient, and a method of delivery of such an implant in or near the heart.

### SUMMARY

The disclosure generally describes a system and a method for delivering to a patient an implant usable for pacing, the implant including a conductive hydrogel formed by in situ mixing of two precursor solutions in in situ curing. The system comprise a delivery tool (e.g., a sheath) and a device. The device is inserted percutaneously to a target location in the vasculature of a patient and anchored. Precursor solutions of a hydrogel are injected via the device into the vasculature of the patient at the target location and cure in situ. The cured hydrogel connects an electrical connector of the device to the tissue of the patient. An electrical connector and the hydrogel are part of the portion of the device that remains implanted in the patient. The electrical connector can then be connected to an external electrically active device (e.g., a pacemaker).

An example intended use of the system is providing therapy for cardiac disease states such as heart failure and arrhythmia. However, the system can generally be utilized in other cases where electrical stimulation from an injected hydrogel is beneficial to a patient. For example, the system can be used for providing therapy through the vasculature to surrounding neural tissue. In other instances, the system could be utilized to provide ablative therapy to specific locations. To achieve this ablative therapy, the hydrogel solution is injected in the target location, and ablative power may be supplied through the connector interface.

Preferably, the system may be characterized by one or more of the following elements:
an implantable shaft system, having a connector, conductive wires, and electrodes, capable of injecting precursor solutions in a patient,
a mixing head capable of mixing the precursor solutions in the patient to form a hydrogel in situ,
an electrically conductive distal electrode in contact with the formed hydrogel, and/or pinching and/or detachment mechanisms to retrieve a portion of the system from the patient and only leave the implant in the patient.

The system may further comprise other elements, such as an occluder assembly to anchor the shaft system at a target location, a detachable container for storing precursor solutions, a catheter hub, and/or a catheter hub for connecting the implant to the detachable container.

### BRIEF DESCRIPTION OF THE DRAWINGS

For a more detailed description of the embodiments of the disclosure, reference will now be made to the accompanying drawings, wherein:

FIG. 1 is a view illustrating the use of a device for delivering an implant to a patient, wherein the implant includes a hydrogel capable of curing in situ, and the implant is usable for pacing;

FIG. 1A is a view illustrating the device shown in FIG. 1;

FIG. 1B is a view illustrating the implant shown in FIG. 1;

FIGS. 2A and 2B are views of a detachable container that is included in the device shown in FIG. 1;

FIG. 3 is a view of a catheter hub that is included in the device shown in FIG. 1;

FIG. 4 are views of a shaft system that is included in the device shown in FIG. 1;

FIGS. 5A and 5B are views of different configurations of an occluder assembly that is included in the device shown in FIG. 1;

FIG. 6 is a view of a mixing head that is included in the device shown in FIG. 1;

FIG. 7 is a sectional view of a joined shaft that is included in the shaft system shown in FIG. 4;

FIG. 8 are views of a combined pinching and detachment mechanism that is included in the device shown in FIG. 1;

FIGS. 9A-9C illustrate the working of the combined pinching and detachment mechanism shown in FIG. 8;

FIGS. 10A-10C illustrate a system with dual occluders.

### DETAILED DESCRIPTION

The disclosure describes a preferred embodiment of an implant which delivers an in situ curing hydrogel to a target location and maintains electrical contact between the delivered hydrogel and an electrically active device, such as cardiac or neural pacing device. The implant may function as a specialized lead capable of delivering in situ mixing hydrogel. The hydrogel, which is capable of curing in situ, may function as an extension coupling the lead to the patient's tissue. The implant comprises of an implantable shaft system. The shaft system comprises of a main shaft and a branched shaft that merge into a joined shaft. The shaft system houses multiple lumens that exit at a mixing head, one or more electrodes and an occluder assembly. The mixing head may be electrically conductive and may form a distal electrode. Each electrode along the shaft system is connected via the branched shaft to a standard pacemaker/defibrillator connector.

A device comprises the implant, a detachable container that holds the precursor solutions, and a catheter hub. The implant is connected to the detachable container via the catheter hub. The device, in a preferred embodiment, will be inserted percutaneously through vasculature to a target location in the heart. An occluder assembly will be deployed to anchor the device at the target location. Precursor solutions will be injected into the target vessel by pressing on a plunger assembly that has been pre-filled with the precursor solutions. When the two precursor solutions meet at a mixing head distal to the occluder assembly, they will mix while filling the vasculature at the target location, and, in time, cure to form a hydrogel. Using a combination of pinching mechanism and detachment mechanism, a portion of the main shaft and the catheter hub of the catheter assembly will be detached and the removed. The pinching mechanism will cap or close the main shaft, and the detachment mechanism will cut a portion of the main shaft. The anchoring sleeve attached distal to the detachment mechanism, still inside body, will be used to suture down the pinching mechanism and/or a remaining portion of the shaft system onto a pocket made in the body. The connector attached a branched shaft can then be connected to a pacemaker to pace through the implant. The connector interface, in certain embodiments, is connected to a ring electrode on the joined shaft proximal to the occluder assembly, and the mixing head which acts as a distal electrode. To achieve this, the mixing head will be, at least in part, comprise an electrically conductive material.

For using the device, an operator identifies a target vessel location in the heart using common imaging techniques. A delivery sheath inserted percutaneously through a vessel such as the subclavian vein or the cephalic vein, is used to deliver a guidewire to the location. The guidewire is fed from the distal end through the length of the branched and main shafts of the shaft system. The device is then snaked through the delivery sheath to the target location. When the device exits the delivery sheath, the occluder assembly is deployed. In certain embodiments, a distal occluder system is deployed, the catheter is pulled back such that the distal tip is located at the proximal end of the system where the hydrogel is to be placed, and a proximal occluder system is deployed. Using the plunger assembly on the detachable container, the precursor solutions are injected through the shaft system into the target vessel through the mixing head. The target vessel will be filled with a prescribed volume of the precursor fluids that, in time, will cure to form a hydrogel. When curing, the hydrogel will be formed around and distal to the mixing head. Combined pinching and detachment mechanisms on the shaft system will be used to detach the main shaft from the catheter hub. Once detached, the catheter hub will be removed. The anchoring sleeve will be pulled up to cover the cut portion of the main shaft and the combined mechanism will be pushed into the anchoring sleeve. The anchoring sleeve will then be sutured onto a pocket made close to the percutaneous entry site. The connector interface is then connected to a pacemaker or defibrillator to provide therapy.

In the event the implant attached to the hydrogel has to be replaced, for example due to migration of the lead or failure of the lead, the occluder assembly will be reset to an undeployed configuration. The distal electrode included in the mixing head and embedded into the hydrogel will be removed by pulling it out or by unscrewing it. When removed, the hydrogel in the vessel may have voids where the mixing head and its mixing features (such as bumps, holes, screw etc.) were present. Once removed, another device as described herein will be placed just proximal to the previously filled hydrogel. Inserting precursor solutions will fill the space beyond the newly inserted device, including the voids in the previously filled hydrogel. The newly inserted device may also mechanically attach to the hydrogel, or to a part of the mixing head or the whole mixing head which may have been detached from the shaft system and left behind. The newly inserted device can then be prepared for use as described earlier.

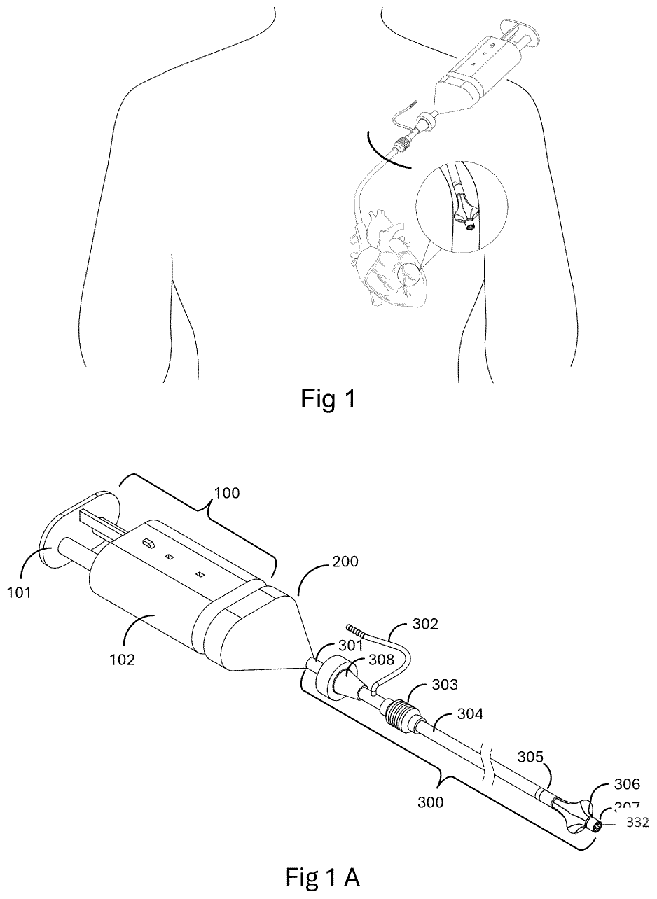

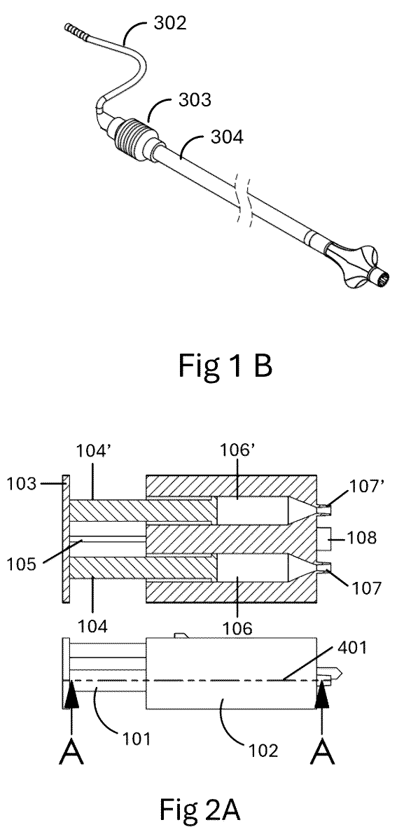

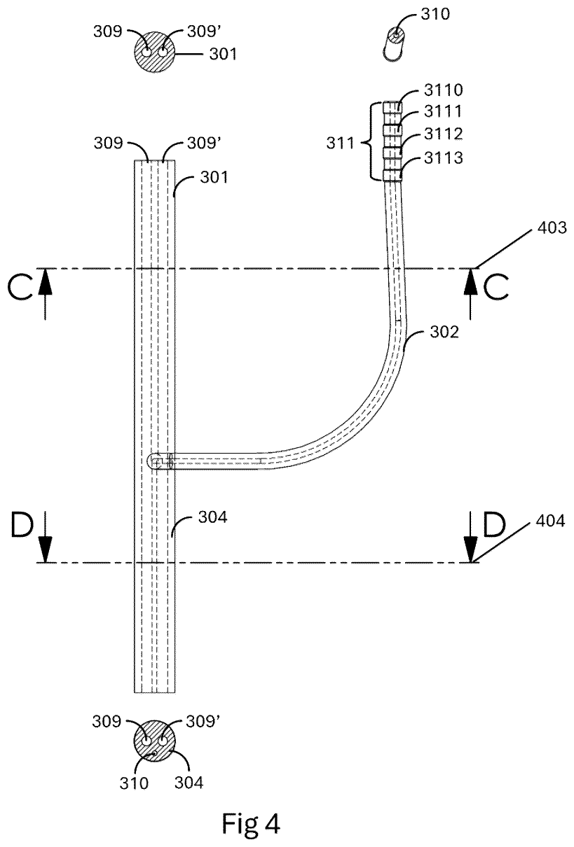

FIG. 1 shows an exemplary embodiment of the device including a detachable container 100, and a catheter assembly. The catheter assembly comprises a catheter hub 200, and a shaft system 300. In certain embodiments, the detachable container 100 contains a plunger assembly 101 that is mechanically controlled, that is, pushed through pre-filled body 102. The detachable container 100 can connect to and interface with the catheter hub 200. The catheter hub 200 provides an interface for fluids such as saline, air, precursor solutions or others to be injected into the shaft system 300. The shaft system 300 comprises a main shaft 301 that meets a branched shaft 302 to form a joined shaft 304. The shaft system 300 further comprises an anchoring sleeve 303 used to anchor the device to the body of a patient once deployed. A connector on a proximal end of the branched shaft 302 is electrically connected to a ring electrode 305 on the joined shaft 304, and to a distal electrode 332 on a mixing head 307. An occluder assembly 306, when deployed, also allows for anchoring the catheter at the target location, for example, to a vasculature wall. The distal electrode 332 comprises an electrically conductive component located at the distal tip of the joined shaft 304. The shaft system 300 also comprises a combined pinching and detachment mechanism 308 to detach the joined shaft 304 from the catheter hub 200. The combined mechanism 308 comprises three concentric parts formed as a twistable screw which when turned in a specific direction, compresses or pinches the main shaft 301, closing the main shaft 301 distal to the combined mechanism 308. Upon further turns of the screw, a cutting edge is engaged proximal to where the main shaft 301 was pinched, releasing the combined mechanism 308 from the branched shaft 302 and the joined shaft 304. FIG. 1A shows an embodiment of the device when injecting the hydrogel for therapy. FIG. 1B shows an embodiment of the implant after it is implanted in the patient. Using the combined mechanism 308, the main shaft 301 has been severed leaving behind the branched shaft 302 and joined shaft 304. The anchoring sleeve 303, the joined shaft 304 and the branched shaft 302 will remain subcutaneously in a pocket where a traditional pacemaker is placed. In certain embodiments, once the main shaft 301 is severed, it is capped off by the combined mechanism 308 and the combined mechanism 308 is attached to or nestled inside the anchoring sleeve 303 (not shown in FIG. 1B). The connector 311 (see FIG. 4) at the proximal end of branched shaft 302 will be connected to a standard pacemaker or an implantable cardioverter defibrillator for providing therapy via electrodes distal to the joined shaft 304. The occluder assembly 306 will remain deployed after implant.

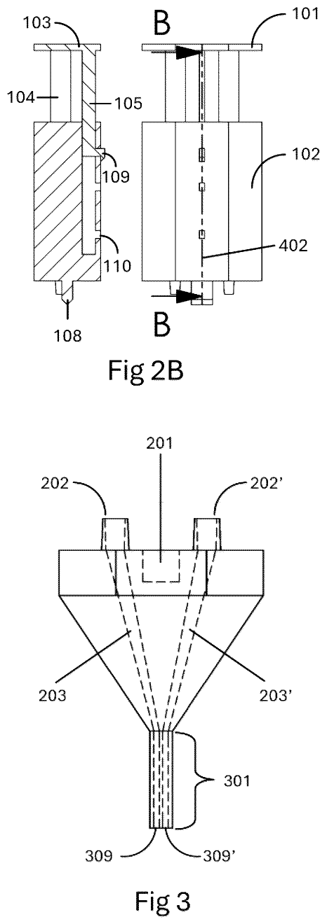

FIGS. 2A and 2B shows sectional views of the detachable container 100. The detachable container 100 comprises a plunger assembly 101. The plunger assembly 101 comprises a handle 103 connected to two plungers 104 and 104′, and a guide 105. When the handle 103 is pushed, plungers 104 and 104′ move through barrel 106 and 106′ respectively. These pre-filled body 102 will expel fluid at an equal rate through outlets 107 and 107′. A fastening feature 108 interfaces with a corresponding fastening feature 201 provided in the catheter hub 200 and locks it into place. When handle 103 is pushed, the guide 105 will pass through channel 110 on the pre-filled body 102 and tooth feature 109 will latch onto ratchet openings at different locations. A separate part can be used to unlatch the guide 105 allowing it to move forward.

FIG. 3 shows a detailed view of the catheter hub 200 attached at its distal tip to the main shaft 301 of shaft system 300. The catheter hub 200 has two inlets 202 and 202′ that interface with outlets 107 and 107′ of the detachable container 100. The inlets 202 and 202′ may provide a standard connection, such as a LUER-LOK™ or Luer-slip connection, the detachable container 100. Hollow lumens 203 and 203′ interface with lumens 309 and 309′ respectively. In certain embodiments, catheter hub 200 may include a mixing chamber into which lumens 202 and 202′ connect so that fluids will mix in this mixing chamber before exiting the hub via a single lumen 309 which extends through shaft 304.

FIG. 4 provides a detailed view of the proximal end of the shaft system 300. A main shaft 301 comprising lumens 309 and 309′, meets with a branched shaft 302, having a single lumen 310 to form a joined shaft 304. The branched shaft 302 terminates at its proximal end at a connector 311. The connector 311 in certain embodiments comprises conductive interfaces 3110, 3111, 3112 and 3113 at the proximal end of the branched shaft 302. In certain embodiments, the proximal end may comprise of a standard pacemaker connector such as an IS-1, IS-4, DF-1 or DF-4 interfaces. Conductors (e.g., including conductive wires) run from these interfaces along the branched shaft 302, and joined shaft 304 to ring electrodes 305 and distal electrode 332.

In certain embodiments, shaft system 300 may not include a separate main catheter shaft, e.g. the combined shaft ends only with the branched shaft 302 and does not include another shaft 301 with which it combines. In such embodiments, catheter hub 200 may be connected to the detachable container 100 on the proximal end, and the distal end of the hub comprises of a design that allows to be slotted into the connector mechanism of the branched shaft 302. For example, if the branched shaft 301 has a proximal IS-4 connector, catheter hub 200 will have a mating component that fits into the connector. The hollow tubing inside catheter hub 200 will be aligned with the hollow lumen of the branched shaft 302 allowing for fluid and other materials to pass through.

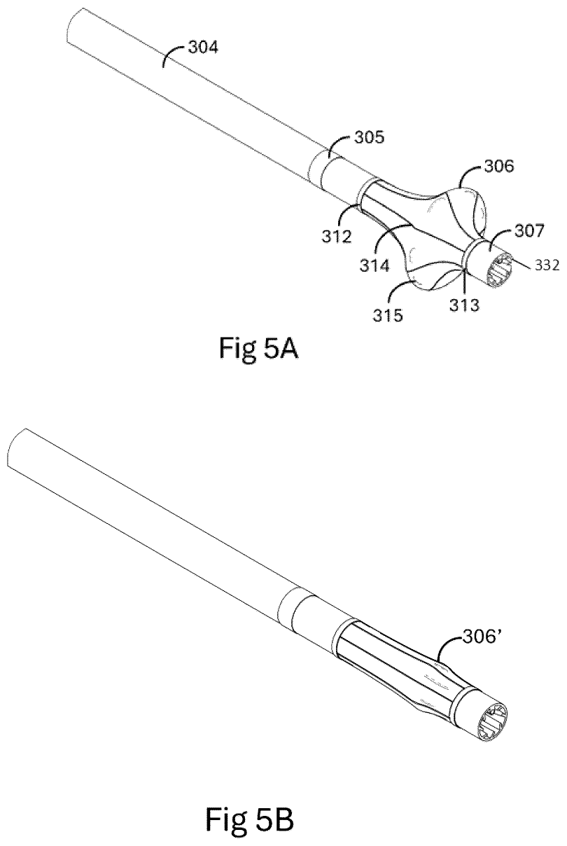

FIGS. 5A and 5B show detailed views of the occluder assembly 306 at the distal end of the shaft system 300. In a preferred embodiment, the occluder assembly 306 is present distal to a ring electrode 305 and in part overlaps and is in contact with the conductive mixing head 307. The occluder assembly 306 comprises two mechanical anchoring rings 312 and 313 placed on the shaft system 300. Elastic spines 314 run from the proximal anchoring ring 312 to the distal anchoring ring 313. Example of materials the elastic spines 314 may comprise of include nitinol, steel, LDPE, or HDPE. The elastic spines 314 support a mesh like material 315 forming the occluder assembly 306. Example of materials the mesh like material 315 may comprise of include polyester fabric, PTFE, Nylon or other materials commonly known in the art. FIG. 5A shows the occluder assembly 306 in a deployed state, and FIG. 5B shows the occluder assembly 306 in an undeployed default state 306′. In certain embodiments the occluder assembly 306 is in its default undeployed state when it is within a delivery tool. When the delivery tool is sufficiently removed, the elastic spines 314 expand, deploying the occluder assembly 306.

In certain embodiments, the combined shaft may include a proximal occlusion mechanism and a distal occlusion mechanism a fixed distance away from the proximal occlusion mechanism. The use of at least one, and preferably two, distinct occluding mechanisms designed to confine the injected hydrogel material to a specific target location within a vessel, preventing leakage and ensuring precise delivery. A proximal occluder may be employed to prevent backflow of the hydrogel relative to the delivery system, while a distal occluder may be utilized to prevent forward flow of the hydrogel material. The overall purpose of these occluders is to confine the precursor solutions to a specific location during injection and curing.

In certain embodiments, the shaft wall in between the two occlusion mechanisms may be perforated to allow for injecting the hydrogel with the perforations interfacing with the inner hollow lumen. When the hydrogel is injected, it will exit through these openings or pores into the target vessel blocked on either end by the occlusion mechanism.

A proximal occlusion mechanism may be typically positioned on the catheter shaft proximal to the mixing mechanism. In a preferred embodiment, this occluder comprises foldable spines, constructed from materials such as Nitinol, steel, LDPE, or HDPE. These spines extend between a proximal anchoring ring and a distal anchoring ring, and support a membrane, for example, made of polyester fabric, PTFE, or Nylon. In its deployed state, the spines are unfolded radially causing the membrane to open and occlude the target vessel. In an undeployed or collapsed state, it is folded. Typically, the occluder is held in this folded state by an external device, such as a delivery sheath covering it. When this sheath is pulled or pushed relative to the catheter, the spines unfurl, transitioning the occluder mechanism to its deployed state. Once implanted, this proximal occluder system is intended to remain in the deployed state.

In other embodiments, the proximal occluder may comprise a deflated balloon, which is placed along the length of the catheter shaft and connected via a lumen to the hub. In its deployed state, the balloon remains inflated with air, saline, or other fluids to occlude the vessel; it can be deflated for catheter retraction if needed, particularly if the distal occluder has already been deployed. In another alternative, the proximal occluder might consist of foldable spines with a covering membrane, held in an undeployed state under tension by a tension wire. When this tension wire, running in a lumen along the catheter shaft and connected to the hub, is pulled or pushed, the spines are released radially, deploying the occluder.

A distal occlusion mechanism is designed to prevent forward flow of the hydrogel beyond the intended injection area. In certain embodiments, the distal occluder comprises an “umbrella-like” design. This design features self-expanding frames, which may be constructed from a shape memory metal such as Nitinol, and a mesh, made from materials like polyester, designed to catch the materials of the precursor solution. In a specific configuration described, this umbrella-like distal mechanism may initially envelop the proximal occluder mechanism. The deployment sequence involves advancing the system within a delivery sheath to the distal location requiring occlusion. Retracting the delivery sheath then allows the proximal occluder to expand; this expansion of the proximal occluder, in turn, causes the enveloping umbrella-like distal mechanism to open and deploy within the target vessel. The distal occluder is designed such that once it is deployed, the delivery sheath can be advanced again to fold or collapse the proximal occluder, allowing the catheter system (now with a deployed distal occluder) inside the delivery sheath to be retracted or repositioned.

Alternatively, the distal occluder may be a separate mechanism deployed initially, prior to advancing the main catheter system of this invention; in this method, the distal occluder acts as a standalone device. In some embodiments, the distal occluder may be tethered to the proximal occluder, or another part of the catheter shaft, by means of a wire, which can be conductive or non-conductive. Before deployment, this tethering wire might be wrapped around the conductive mixing head at the distal tip of the catheter or around the distal end of the catheter shaft. In yet another alternative embodiment, the distal occluder may comprise a gel or gel-like material that is delivered to tamponade the vessel distally. This gel may be resorbable by the vessel over time. The gel could be present at the tip of a wire, which in some embodiments is preloaded into the catheter, or in other embodiments, the wire carrying the gel is a separate unit inserted into the catheter. In such a case, the catheter is first placed at the distal location needing occlusion, and then the wire with the gel is advanced. Upon exiting the catheter, the preloaded gel expands and adheres to the vessel walls, thereby occluding it. The main catheter can then be retracted to a more proximal location from where the therapeutic hydrogel will be delivered into the now distally-occluded segment.

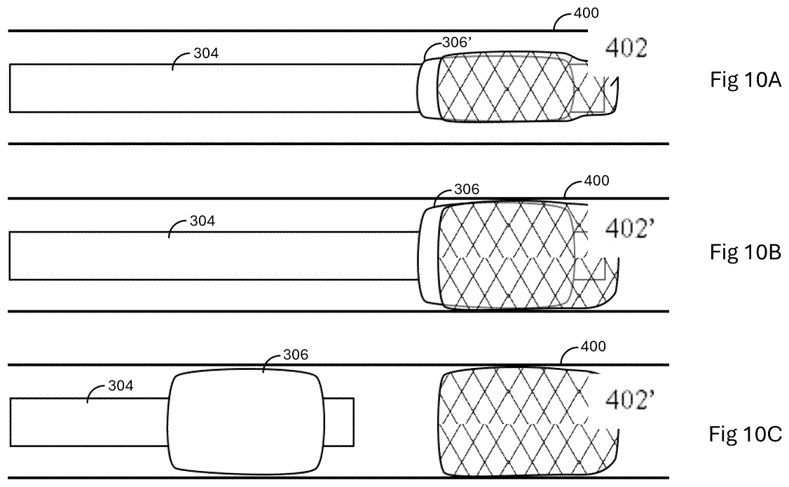

FIGS. 10A-10C show a detailed view of a distal occlusion device on the hydrogel delivery catheter. A collapsed wire basket with fabric mesh 402 is placed on the end of the hydrogel delivery catheter such that it covers the occluder assembly 306 and the mixing head 307. The catheter is then guided to the desired location, distal to where the hydrogel will be delivered, in the blood vessel 400. When the occluder mechanism 306 is activated expanding the wire basket with fabric mesh into its expanded state 402′. The occluder mechanism is then retracted and the catheter is moved proximally up the vessel. The occluder mechanism 306 is then once again activated to occlude the proximal side of the vessel 400. This occludes both the proximal and distal portions of the vessel for hydrogel delivery. In other embodiments the wire basket with fabric mesh 402 is connected to the catheter shaft 304 with the use of a wire and may be used for electrical stimulation.

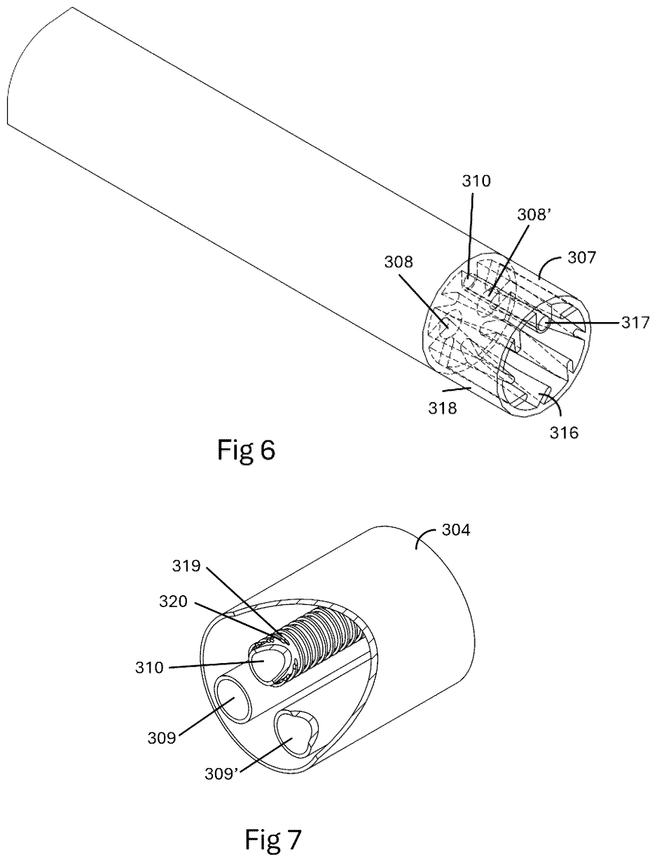

FIG. 6 shows the mixing head 307 at the distal tip of the shaft system 300. The mixing head 307 comprises mixing features present along the inner wall 316, an outer wall 318 and an opening 317 for passing fluids or instruments through. Single lumen 310 within the joined shaft 304 interfaces with an opening 317 in the mixing head 307. Guidewires, stylets, or fluids such as saline or contrast entering the single lumen 310 at the branched shaft 302 exits through the opening 317. Lumens 309 and 309′ traversing through the joined shaft 304 exits into the inner wall 316 of the mixing head 307 where mixing features are present. In certain embodiments, the mixing features comprise baffles along the inner wall 316 of the mixing head 307. The inner and outer walls 316 and 318 of the mixing head 307 may be treated to improve adhesion to the hydrogel and increase surface area of contact for better energy transfer. In certain embodiments, all surfaces of the mixing head 307 will comprise of an electrically conductive material such as platinum iridium or stainless steel. In certain embodiments, either the outer surface 318 or inner surface 316 will comprise of an electrically conductive material; the other surface will be made up of a non-conductive material. In certain embodiments, at least a portion of the mixing head 307 will comprise of non-conductive material. In certain embodiments, mixing features present in the inner wall 316 may be a separate part interfacing with the inner wall 316. Other mixing features may include holes, protrusions, or a helical screw design.

FIG. 7 shows a broken-out section of the joined shaft 304. Single lumen 310 is surrounded by co-radially wound conductors 319 and 320 that connects the connector 311 to ring electrodes 305 and distal electrode 332. Conductors 319 and 320 may be connected to one of more conductive interfaces 3110, 3111, 3112 and/or 3113.

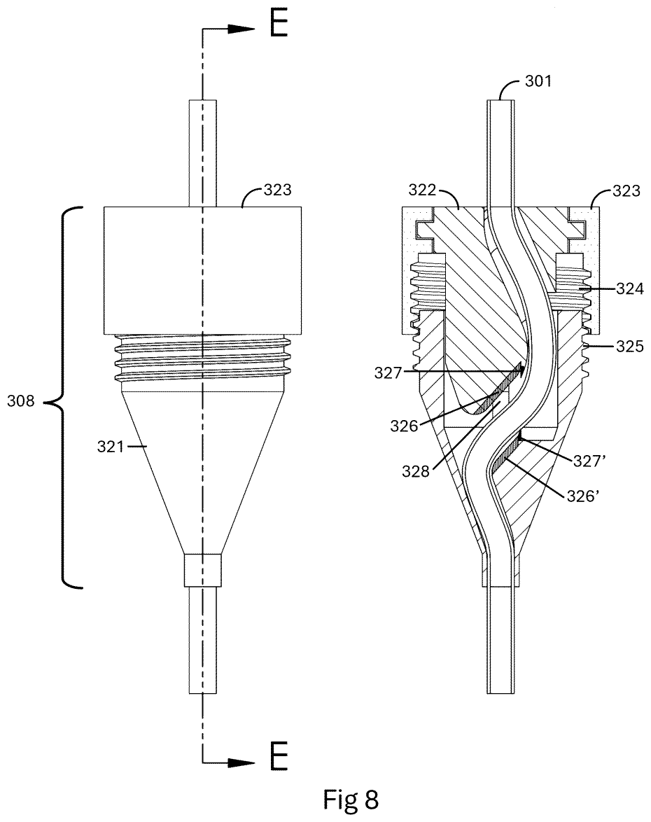

FIG. 8 shows a detailed view of the combined pinching and detachment mechanism 308. There are two jaw members 321 and 322, through which the main shaft 301 passes. A twistable screw or cap 323 is turned to engage the threads 325 of the jaw member 321 with the threads 324 of the twistable screw or cap 323. With the jaw member 321 fixed in position over the main shaft 301, twisting the twistable screw or cap 323 moves the jaw member 322 closer to the jaw member 321. The main shaft 301 will be progressively pinched between flat surfaces 326 and 326′; these flat surfaces are fixed to 322 and 321 on either side of the main shaft 301. Further twisting 323 will engage the cutting edges 327 and 327′ located on either side of the main shaft 301 and fixed to the jaw members 322 and 321, cutting the main shaft 301 and separating everything proximal to the combined mechanism 308. There is a guiderail 328 located between the jaw members 321 and 322 which prevents rotation of these jaw members 321 and 322 relative to one another while twisting the twistable screw or cap 323. In certain embodiments, the twistable screw or cap 323 may comprise of a lock mechanism that directly engages with the jaw members 321 and 322 by pushing or compressing the two jaw members 321 and 322 together, which would lock after sufficient engagement is achieved, compressing the main shaft 301 distal to the cutting edge; this will sever the mains shaft 301 and release the catheter hub 200 and other proximally located parts of the device.

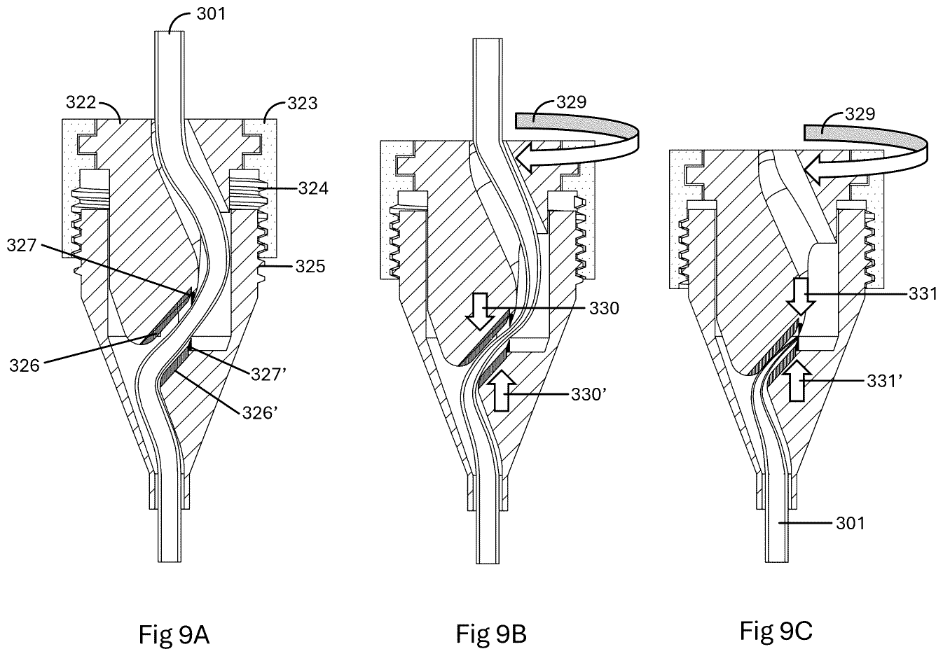

FIGS. 9A-9C show a detailed view of the working of combined mechanism 308. When a twistable cap or screw 323 with its threads 324 is turned (arrow 329 shows motion) to engage with the threads 325 of the jaw member 321, the jaw member 322 moves downwards. (FIG. 9B) As a result of this motion, the main shaft 301 will be progressively pinched between flat surfaces 326 and 326′ (arrows 330 and 330′ shows motion of jaw members 321 and 322). (FIG. 9C) Further twisting the twistable cap or screw 323 will engage the cutting edges 327 and 327′ severing the main shaft 301 and separating everything proximal to the combined mechanism 308 (arrows 331 and 331′ shows motion of cutting edges 327 and 327′).

In addition to the foregoing, the disclosure also contemplates at least the following embodiments 1-27. It should be noted that any element of any of embodiments 1-27 may further include details related to this element that are disclosed in a paragraph or figure describing the preferred embodiments without necessarily including details of other elements that are disclosed in the same or other paragraph or figure.

### Embodiment 1

Embodiment 1 is an implant that comprises a shaft system having an intermediate portion, an anchoring sleeve, a mixing head, and an occluder assembly.

The shaft system includes a connector, conductive wires, and electrodes. The conductive wires couple the connector to each of the electrodes. For example, at least some of the conductive wires may be co-radially coiled along a portion of the length of the shaft system. The connector is attached to a proximal end of the shaft system. The electrodes are attached to the shaft system.

The shaft system houses internal lumens. A hydrogel is formed by curing a mixture of solutions that have flown separately in some of the lumens and are discharged and mixed in the mixing head. At least one of the electrodes, which is referred herein as the distal electrode, is located such that the hydrogel is formed in contact with the distal electrode.

In an example use, the connector may be configured to be coupled to a pacemaker. The connector may be a standard pacemaker connector such as an IS-1, IS-4, DF-1 or DF-4 connector. Thus, the pacemaker may be capable of delivering an electrical stimulus to a patient via the connector, the conductive wires, the electrodes, and the cured hydrogel. For example, the stimulus may be delivered to the patient's heart. The cured hydrogel may be used to increase the surface area for providing the stimulus.

The anchoring sleeve and the occluder assembly are placed on the shaft system so that the shaft system passes through the anchoring sleeve and the occluder assembly. The mixing head is adjacent to a distal end of the shaft system. The occluder assembly is placed next and proximal to the mixing head. The anchoring sleeve is placed on the intermediary portion of the shaft system.

The anchoring sleeve has undeployed and deployed configurations, wherein, in the deployed configuration, the anchoring sleeve contacts subcutaneous tissue of a patient so that the implant remains in place at a target location in the patient vasculature.

The occluder assembly is used to prevent leakage of the mixture of solutions and/or to confine this mixture to a specific location. In particular, the occluder assembly can be used to prevent backflow of the mixture of solutions toward the proximal end of the implant or forward flow past the point at which the hydrogel is desired to be placed.

The mixing head, or mechanism, includes a mixing cavity with mixing features to promote the mixing of solutions in situ (i.e., in the body of the patient) after the solutions are discharged from some of the lumens internal to the shaft system into the mixing head.

### Embodiment 2

Embodiment 2 is an implant as described in embodiment 1, wherein the shaft system comprises a main shaft section, and a branched shaft that meets the main shaft to form a joined shaft. The intermediate portion of the shaft system consists of the main shaft. The distal end of the joined shaft is the distal end of the shaft system. The proximal end of the shaft system is the proximal end of the joined shaft.

The main shaft section has at least a pair of lumens that extend through the joined shaft to the mixing head. The branched shaft has at least one lumen that extends through the joined shaft to the mixing head and houses the conductive wires.

The mixing head has an opening along its outer surface or an inner tube that are in communication with this at least one lumen and are isolated from the mixing cavity. The opening or inner tube may also be used to pass the fluids such as saline or contrast, a guidewire, a stylet, or other devices through the mixing head while avoiding the mixing cavity. The at least one lumen within the branched shaft exits through the mixing head. This at least one lumen may also be used to pass the fluids, the guidewire, the stylet or the other devices through the shaft system.

The connector is located at the proximal end of the branched shaft. The mixing head is adjacent to the distal end of the joined shaft. The electrodes are connected to the connector via the conductive wires that run along the length of the branched shaft and the joined shaft.

### Embodiment 3

Embodiment 3 is an implant as described in embodiment 2, wherein the branched shaft and at least a portion of the joined shaft may comprise an insulating coating. For example, the insulating coating may be made of materials commonly used in pacemaker leads, such as polyurethane, silicone, or other materials known in the art. In addition to the distal electrode, one of the electrodes is a ring electrode. Preferably, the ring electrode is provided on the joined shaft, preferably adjacent and proximal to the occluder assembly. The ring electrode may alternatively be provided on the occluder assembly such that it contacts a wall of the patient vasculature, when the occluder assembly is in the deployed configuration. There may be more than one ring electrode provided along the length of the implant.

### Embodiment 4

Embodiment 4 is an implant as described in any of embodiments 1 to 3, wherein at least a portion of the shaft system, for example a portion of the joined shaft, has a pre-formed bend designed to ensure that an electrode located on it, for example a ring electrode, contacts a wall of the patient vasculature. Pre-formed bend may be made as described in U.S. Pat. No. 7,313,444, which is incorporated by reference herein.

In cases where more than one electrode is provided along the shaft system, more than one pre-formed bend may be present along the shaft system, and each electrode may be located in a pre-formed bend.

### Embodiment 5

Embodiment 5 is an implant as described in any of embodiments 1 to 4, wherein the occluder assembly comprises elastic spines that are together holding a membrane. The membrane may be made of a mesh-like material. When the spines are unfolded radially, the membrane opens and can occlude the patient vasculature at a target location.

The occluder assembly is held in the undeployed (e.g., folded) configuration by a delivery tool, such as a sheath covering the occluder assembly. When the sheath is pulled or pushed, the spines can unfurl and the occluder assembly can expand to a deployed configuration. Once implanted, the occluder assembly is kept in the deployed configuration.

### Embodiment 6

Embodiment 6 is an implant as described in any of embodiments 1 to 4, wherein the occluder assembly comprises a balloon that is deflated in an undeployed configuration. In a deployed configuration, the balloon remains inflated.

A lumen runs along the length of the shaft system for flowing fluid, such as air, saline or other fluids to inflate the balloon.

### Embodiment 7

Embodiment 7 is an implant as described in any of embodiments 1 to 4, wherein the occluder assembly comprises foldable spines with a membrane covering the spines. In an undeployed configuration, the foldable spines are held folded under tension with a tension wire. When the tension wire is pulled or pushed, the spines are released radially and the occluder assembly can expand to a deployed configuration.

The tension wire runs in a lumen provided along the length of the shaft system.

### Embodiment 8

Embodiment 8 is an implant as described in any of embodiments 1 to 7, wherein the mixing head comprises a body having an essentially cylindrical outer wall and an essentially cylindrical, co-axial inner wall forming the mixing cavity. Preferably, the mixing features comprise baffles protruding along the inner wall of the body to promote the mixing of solutions. Instead of, or in addition to the baffles, the inner wall of the mixing head may be provided with bumps, holes, or other known features usable for promoting the mixing of solutions, such as illustrated in U.S. Pub. No. 2022/0305234, which is incorporated by reference herein. Optionally, the mixing features may include an actuator capable of generating vibrations to promote mixing.

If the mixing head has an opening along its outer surface or an inner tube isolated from the mixing area, no mixing feature may be placed in the opening or the inner tube to allow for the unobstructed passage of a stiff device such as a guidewire or a stylet through the mixing cavity. Alternatively, the mixing features may be made of a soft, flexible material, such as a plastic, silicone or a hydrogel, to again allow for the unobstructed passage of a stiff device such as a guidewire or a stylet through the mixing cavity.

### Embodiment 9

Embodiment 9 is an implant as described in any of embodiments 1 to 8, wherein the mixing head comprises an electrically conductive material. For example, the mixing head may be made of stainless steel, platinum iridium, titanium and/or other metals. The distal electrode is formed by the surface of the electrically conductive material. The occluder assembly is preferably attached adjacent to the mixing head so that at least a portion of the occluder assembly is in electrical contact with the electrically conductive material.

### Embodiment 10

Embodiment 10 is an implant as described in embodiment 9, wherein the mixing head is made of electrically conductive materials entirely.

Alternatively, at least a portion of the mixing head may be made of a non-conductive material, such as plastics (e.g., PEBAX, PEEK), polyurethane, polyester, or flexible materials such as silicone. For example, the mixing head may comprise non-conductive mixing features attached to a wall of a conductive body, and a surface of the conductive body may form the distal electrode. Or the mixing head may comprise conductive mixing features attached to a wall of a non-conductive body, and a surface of the conductive mixing features may form the distal electrode. Or the body of the mixing head and the mixing features may be made of non-conductive materials entirely, and the mixing head may comprise a ring electrode on its inner and/or outer surface that forms the distal electrode.

### Embodiment 11

Embodiment 11 is an implant as described in any of embodiments 1 to 10, wherein the distal electrode is formed by a ring electrode proximal to a non-conductive mixing head, or the distal electrode is formed by an interface fastening the shaft system to the mixing head, or the distal electrode is formed by a deployable screw, such as known in the art for active fixation pacemaker leads. The deployable screw may be placed concentrically around, or distal to, the mixing head.

### Embodiment 12

Embodiment 12 is an implant as described in any of embodiments 1 to 11, wherein the surface(s) of the mixing head is (are) modified (e.g., machined to increase roughness, coated with a chemical bonding to metal and the hydrogel) to promote adhesion to the cured hydrogel. For example, any or all surfaces of the mixing head may be treated to improve mechanical or electrical contact. Any or all surfaces of the mixing head may be chemically treated to functionalize the hydrogel onto the mixing head.

### Embodiment 13

Embodiment 13 is an implant as described in any of embodiments 1 to 12, further comprising a pinching mechanism.

The pinching mechanism is placed on the shaft system so that the shaft system passes through the pinching mechanism. The pinching mechanism is attached to the intermediary portion of the shaft system. The anchoring sleeve is placed next and distal to the pinching mechanism. The pinching mechanism is configured to close or seal off the lumens in the intermediary portion of the shaft system.

For example, in the implant as described in any of embodiments 2 to 12, the main shaft section spans between a pinching location, where the lumens of the main shaft are closed or sealed off by the pinching mechanism, and a junction in the shaft system, where the branched shaft meets the main shaft. The pinching location, where the lumens of the main shaft are closed or sealed off, is preferably adjacent to the junction in the shaft system, where the branched shaft meets the main shaft.

### Embodiment 14

Embodiment 14 is an implant as described in embodiment 13, wherein the pinching mechanism comprises two jaw members, a guiderail, and a lock. Each of the two jaw members has a slanted compression surface. The two jaw members are aligned, the slanted compression surfaces face each other, and a space is formed between the slanted compression surfaces. The guiderail maintains alignment and may also prevent relative rotation between the two jaw members. The intermediary portion of the shaft system passes through each of the two jaw members and in the space between them so that the two jaw members are located on opposite lateral sides of the intermediary portion of the shaft system.

When the implant was delivered, the two jaw members have moved closer together, compressing and kinking the intermediary portion of the shaft system. In particular, the two jaw members have moved closer together by a distance sufficient to ensure that the lumens in the intermediary portion of the shaft system are sufficiently pinched to be closed or sealed off. For example, the pinching mechanism was pushed to compress the two jaw members together.

The lock has secured the two jaw members in place when the lumens in the intermediary portion of the shaft system were closed or sealed off.

### Embodiment 15

Embodiment 15 is an implant as described in embodiments 14, wherein the pinching mechanism includes a dial, screw, or cap. The dial, screw, or cap surrounds both jaw members and engages both jaw members so that, with the two jaw members, it forms a set of three concentric parts. The dial, screw, or cap is connected to one jaw member by a screw joint and to the other jaw member by a hinge joint.

When the implant was delivered, twisting the dial, screw, or cap has moved the two jaw members closer together, compressing and kinking the intermediary portion of the shaft system. Preferably, friction between the dial, screw, or cap and the two jaw members is sufficient to prevent untwisting of the dial, screw, or cap, thus securing the two jaw members in place when the lumens in the intermediary portion of the shaft system were closed or sealed off. Alternatively, an additional friction mechanism may further prevent untwisting of the dial, screw, or cap.

### Embodiment 16

Embodiment 16 is an implant as described in any of embodiments 1 to 13, wherein the pinching mechanism comprises a valve that surrounds the intermediary portion of the shaft system.

When the implant was delivered, the valve has caused the pinching the intermediary portion of the shaft system when rotated until the lumens in the intermediary portion of the shaft system was sufficiently pinched to be closed or sealed off.

### Embodiment 17

Embodiment 17 is an implant as described in any of embodiments 13 to 16, wherein the pinching mechanism can be connected to the anchoring sleeve and/or sutured onto the body.

Preferably, the pinching mechanism is sized to be tucked inside or at least partially inserted into the anchoring sleeve. Alternatively or additionally, the pinching mechanism may be fastened to the anchoring sleeve once the pinching mechanism is placed in close proximity to the anchoring sleeve.

### Embodiment 18

Embodiment 18 is an implant as described in any of embodiments 1 to 17, further comprising a detachment mechanism. The detachment mechanism is placed on the shaft system so that the shaft system passes through the detachment mechanism. The detachment mechanism is attached to the intermediary portion of the shaft system. The detachment mechanism includes a sharp cutting edge that is movable.

For example, in any of embodiments 13 to 17, the detachment mechanism is placed next and proximal to the pinching mechanism on a side of the pinching location opposite to the anchoring sleeve.

When the implant is delivered, movement of the sharp cutting edge has caused the cutting of the end of the intermediary portion of the shaft system. For example, the shaft system was cut at a location adjacent and proximal to the pinching location.

For example, the sharp cutting edge may include two blades that were placed on opposite lateral sides of the intermediary portion of the shaft system and that were pushed toward each other. Alternatively, the sharp cutting edge may include an iris-shaped blade that was placed around the intermediary portion of the shaft system and that was closed on the intermediary portion of the shaft system.

### Embodiment 19

Embodiment 19 is an implant as described in embodiment 18, wherein the pinching mechanism and the detachment mechanism are combined into a single mechanism so that movement of the single mechanism sequentially starts pinching and then cutting the end of the intermediary portion of the shaft system.

For example, in the implant as described in embodiments 14 or 15, each of the two blades may be mounted on one of the two jaw members. When the implant was delivered, the movement of the two jaw members toward each other has first compressed the intermediary portion of the shaft system distal to the two blades, and then the two blades have sectioned the proximal part of the shaft system while continuing to compress the intermediary portion of the shaft system.

### Embodiment 20

Embodiment 20 is a device that comprises a detachable container for storing precursor solutions, and a catheter assembly. The catheter assembly comprises an implant as described in any of embodiments 1 to 19 (with the intermediary portion of the shaft system/main shaft being un-sectioned), and a catheter hub.

The catheter hub is adjacent to the intermediary portion of the shaft system. The catheter hub couples the detachable container to lumens housed internally in the shaft system to allow each solution pre-filled in the detachable container (e.g., one of a plurality of hydrogel precursors) to flow through one of the lumens housed internally in the shaft system, and toward the mixing head.

In use, the proximal end of the intermediary portion of the shaft system is sectioned distal of the catheter hub. For example, if the implant is described in embodiments 18 or 19 (i.e., a detachment mechanism is included in the implant), the intermediary portion of the shaft system may be sectioned using the detachment mechanism. If the implant is described in any of embodiments 1 or 17 (i.e., a detachment mechanism may not be included in the implant), the intermediary portion of the shaft system may have markings indicating where to cut or detach the implant. The intermediary portion of the shaft system may be sectioned using a tool such as a scalpel, a heated blade, scissors or other known cutting tool. In any case, the proximal end of the intermediary portion of the shaft system that has been sectioned, the catheter hub, and the detachable container, may be retrieved so that only the implant remains in the body of the patient.

### Embodiment 21

Embodiment 21 is a device as described in embodiment 20, wherein the implant is described in any of embodiments 1 or 17 (i.e., a detachment mechanism may not be included in the implant), and wherein the device further comprises a removable detachment mechanism.

The removable detachment mechanism is placed on the shaft system so that the shaft system passes through the detachment mechanism. The removable detachment mechanism is attached to the intermediary portion of the shaft system. The removable detachment mechanism includes a sharp cutting edge that is movable. A lever or button is configured to actuate the sharp cutting edge when pressed, and section the intermediary portion of the shaft system. The sectioning releases the proximal end of the intermediary portion of the shaft system, the removable detachment mechanism attached to said proximal end, the catheter hub also attached to said proximal end, and the detachable container, from the implant.

### Embodiment 22

Embodiment 22 is a device as described in embodiments 20 or 21, wherein the implant is described in any of embodiments 13-19 (i.e., a pinching mechanism is included in the implant).

In use, the proximal end of the intermediary portion of the shaft system is sectioned proximal of the pinching location. The catheter hub and the detachable container may then be retrieved so that only the implant remains in the body of the patient.

### Embodiment 23

Embodiment 23 is a device as described in any of embodiments 20 to 22, wherein the detachable container comprises a plunger assembly and a body. The body has a plurality of separate barrels, for example two barrels, that can be pre-filled with precursor solutions that, when mixed, cure and form the hydrogel. For example, the two barrels may be pre-filled with dehydrated precursor powders. The powders may be rehydrated using saline either injected or sucked into the barrels. Each barrel communicates with a protruding outlet at a distal end each barrel. The plunger assembly is inserted into a proximal end of the barrels. The plunger assembly comprises a plurality of plungers, for example two plungers. Each plunger of the plurality is inserted into a corresponding barrel. Some or all the plungers are connected at a proximal end of the plunger assembly to a handle.

The plunger assembly is preferably configured to be pushed or pulled manually directly (e.g., by manipulating the handle). Alternatively, the plunger may be configured to be pushed or pulled using an actuator. The actuator may include a rotatable screw sitting on the handle of the plunger assembly. When turned, the screw pushes the plunger assembly forward or pulls the plunger assembly backward. Alternatively, the actuator may include a syringe pump, or a pneumatic actuator using air pressure.

### Embodiment 24

Embodiment 24 is a device as described in embodiment 23, wherein the plunger assembly includes a guide with a tooth feature that interfaces with a channel in the body. The tooth feature can latch into ratchet openings provided along the channel. The tooth feature can be unlatched, allowing the plunger assembly to travel further. As such, the detachable container is configured to stop the travel of the plunger assembly after a desired distance is traveled and specific volumes of precursor solutions are delivered.

### Embodiment 25

Embodiment 25 is a device as described in embodiments 23 or 24, wherein the protruding outlets include a valve, such as a cross-cut valve, and/or the protruding outlets form a standard syringe connection such as a LUER-LOK™ or a Luer-slip connection

### Embodiment 26

Embodiment 26 is a device as described in any of embodiments 20 to 25, wherein the catheter hub comprises two or more recessed inlets configured to connect to the protruding outlets located at the distal end of each barrel of the detachable container, two or more lumens which allow for fluids (gases or liquids) to flow from the detachable container, through the catheter hub, into the shaft system of the catheter assembly, and a fastening feature that mates with a corresponding fastening feature on the detachable container. Additional lumens may be used to introduce devices (guidewires) into the shaft system of the catheter assembly.

For example, the fastening feature on the catheter hub and/or the corresponding fastening feature on the detachable container include a stopcock, a lip and groove, snap hook and groove, or other known pairs of fastening features.

### Embodiment 27

Embodiment 27 is a method of delivering an implant comprising the steps of providing a device as described in any of embodiments 20 to 26, using the device to deliver hydrogel precursors in the vasculature of a patient, mixing the hydrogel precursors to form a mixture in the vasculature of the patient, curing the mixture to form a hydrogel in the vasculature of the patient, and delivering an electrical stimulus to the tissue of the patient at least in part through the hydrogel.

### Embodiment 28

Embodiment 28 is a method as described in embodiment 27, further comprising replacing the implant attached to the hydrogel.

For example, the occluder assembly is reset to an undeployed configuration; the distal electrode included in the mixing head and embedded into the hydrogel is removed, such as by pulling it out or by unscrewing it; and the implant is removed. The implant is removed. However, the hydrogel previously cured in the vessel remains in place. Once the implant is removed, another device as described in any of embodiments 20 to 26 is delivered proximal to the previously cured hydrogel. Usually, the hydrogel previously cured in the vessel has voids where the original mixing head and its mixing features (such as bumps, holes, screw etc.) were present. Therefore, additional hydrogel precursors are preferably injected. Injecting additional precursor solutions is used to fill the space beyond the newly inserted device, including the voids in the previously filled hydrogel. The newly inserted device may also mechanically attach to the hydrogel, or to a part of the mixing head or the whole mixing head which may have been detached from the shaft system and left behind. The newly inserted device can then be prepared for use as described earlier.

Specific embodiments of the invention are shown by way of examples in the drawings and description. It should be understood, however, that the drawings and detailed description thereto are not intended to limit the invention to the particular form disclosed, but on the contrary, the intention is to cover all modifications, equivalents, and alternatives falling within the scope of the disclosure.

What is claimed is:

1. An implant comprising:
a shaft system including a conductive wire and an internal lumen, wherein the internal lumen and the conductive wire extend to the distal end of the shaft system;
a proximal connector disposed at a proximal end of the shaft system;
an electrode disposed at a distal end of the shaft system;
an anchoring sleeve disposed at an intermediate portion of the shaft system;
a mixing head disposed at a distal end of the shaft system, wherein the mixing head is coupled to the internal lumen; and
an occluder assembly coupled to the shaft system between the anchoring sleeve and the mixing head.

2. The implant of claim 1, wherein the mixing head is configured to mix material supplied through the internal lumen and place the mixed material in contact with the electrode after the implant is placed in a patient.

3. The implant of claim 2, wherein the mixed material forms an electrically conductive hydrogel.

4. The implant of claim 3, wherein the mixing head is electrically conductive and in electrical contact with the electrode and the hydrogel.

5. The implant of claim 1, wherein the conductive wire is operable to connect to a pacemaker.

6. The implant of claim 1, wherein the anchoring sleeve has a deployed configuration wherein the anchoring sleeve contacts subcutaneous tissue to affix the implant in place in a patient.

7. The implant of claim 1, wherein the occluder assembly is configured to prevent backflow of material from the mixing head away from the distal end of the shaft system toward the proximal end.

8. The implant of claim 1, further comprising a branched shaft coupled to the shaft system between the connector and the anchoring sleeve, wherein the conductive wire extends through the branched shaft and terminates at one or more conductive interfaces.

9. The implant of claim 1, wherein the connector is configured to detachably couple a container body to the internal lumen.

10. The implant of claim 1, further comprising a pinching mechanism operable to close and seal the internal lumen at the proximal connector.

11. A method for providing electrical stimulus comprising:
providing an implant including,
a shaft system with a conductive wire and an internal lumen, wherein the internal lumen and the conductive wire extend to the distal end of the shaft system;
a proximal connector disposed at a proximal end of the shaft system;
an electrode disposed at a distal end of the shaft system;
a mixing head disposed at a distal end of the shaft system, wherein the mixing head is coupled to the internal lumens; and
an occluder assembly coupled to the shaft system between the anchoring sleeve and the mixing head;
inserting the implant into a patient's vasculature;
deploying the occluder assembly to engage the patient's tissue;
delivering hydrogel precursors through the internal lumen to the mixing head;
mixing the hydrogel precursors with the mixing head so as to form a conductive hydrogel in contact with the patient's vasculature and the electrode;
activating a pinching mechanism to close and seal the internal lumen at the proximal connector;
activating the anchoring sleeve to couple the implant to the patient at a desired location;
coupling the conductive wire to an electrically active device; and
providing an electrical stimulus from the electrically active device to the patient.

12. The method of claim 11, wherein the implant further includes a container body detachably coupled to the internal lumen.

13. The method of claim 12, wherein actuating the pinching mechanism detaches the container body from the implant.

14. The method of claim 11, further comprising:
resetting the occluder assembly to an undeployed position;
detaching the electrode from the conductive hydrogel;
removing at least a portion of the shaft system including the electrode from the patient without removing the conductive hydrogel; and
coupling a new electrode to the conductive hydrogel.

15. An implant comprising:
means for supplying precursor solutions of a conductive hydrogel;
means for mixing the precursor solutions to form a conductive hydrogel at a selected location within a patient's vasculature; and
means for electrically coupling the conductive hydrogel to an electrically active device.

16. The implant of claim 15, wherein the means for supplying precursor solutions includes means to detach the implant from a supply of precursor solutions.

17. The implant of claim 16, wherein the means to detach the implant includes a pinching mechanism to close and seal the means for supplying precursor solutions.

18. The implant of claim 15, further comprising means for attaching the implant to the patient.

19. The implant of claim 15, further comprising a means to occlude the movement of the conductive hydrogel through the patient's vasculature.

20. The implant of claim 15, wherein the means to occlude is adjustable between a deployed position and an undeployed position.
